(19) United States
(12) Patent Application Publication
(10) Pub. No.: US 2026/0146934 A1
(43) Pub. Date: May 28, 2026

(54) OPTICAL SEDIMENT TRAP SENSOR AND WIPER ASSEMBLY

(71) Applicant: Sequoia Scientific, Inc., Bellevue, WA (US)

(72) Inventors: Wayne Homer Slade, SR., Vero Beach, FL (US); Henry Charles Pottsmith, Bellevue, WA (US)

(21) Appl. No.: 19/397,655

(22) Filed: Nov. 21, 2025

Related U.S. Application Data
(60) Provisional application No. 63/724,610, filed on Nov. 25, 2024.

(51) Int. Cl. G01N 15/06; B08B 1/14; G01N 33/18

(52) U.S. Cl. CPC G01N 15/0612; B08B 1/143; G01N 33/1886

(57) ABSTRACT

In accordance with one embodiment of the present disclosure, a sensor assembly for detecting carbon particles within water is provided. The sensor assembly can include a source portion for generating an optical beam and a detect portion for receiving the optical beam. The detect portion can having a housing that defines a detection surface, which can be configured to receive carbon particles. The detection surface can have an area of at least 5 cm squared. The sensor assembly can also include a wiper member coupled to the detect portion. The wiper member ca be movably coupled to the detection portion such that the wiper member can move between at least a first position, where the wiper member surrounds the detection surface, and a second position, where the wiper member is spaced apart from the detection surface.

### CROSS-REFERENCE TO RELATED APPLICATIONS

The present application claims the benefit of, and priority to, U.S. Provisional Ser. No. 63/724,610 , filed Nov. 25, 2024, entitled “OPTICAL SEDIMENT TRAP SENSOR AND WIPER ASSEMBLY”, the contents of which are hereby incorporated by reference in their entirety.

### BACKGROUND

The oceanic carbon cycle refers to how carbon can flow between the ocean and other portions of the Earth, such as the atmosphere. There are a few main ways carbon can interact with the ocean, one of which is referred to as the biological carbon pump. Generally speaking, the biological carbon pump refers to the ocean's biological process of storing carbon. One aspect of this biological carbon pump can involve carbon that passively sinks within the ocean. This process can involve marine life, such as phytoplankton, absorbing carbon during its life and sinking towards the ocean floor after dying, taking the absorbed carbon with it.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a submersible system is described. The submersible system can include a submersible vehicle having a housing and a sensor assembly coupled to the housing of the submersible vehicle. The sensor assembly can include a first housing portion that defines a detection surface for collecting particles. The detection surface can have an area of at least 5 cm squared.

In accordance with another embodiment of the present disclosure, a sensor assembly is described. The sensor assembly can include a source portion having a housing and a detect portion having a housing spaced from the housing of the source portion. The source portion can be configured to generate an optical beam. The housing of the detect portion can define a detection surface for receiving the generated optical beam, the detection surface having an area of at least 5 cm squared.

In accordance with another embodiment of the present disclosure, a method of operating a sensor assembly is described. The method can include deploying the sensor assembly. The sensor assembly can include a source portion having a housing and a detect portion having a housing spaced from the housing of the source portion. The source portion can be configured to generate an optical beam and the detect portion can define a detection surface for receiving the optical beam. The detection surface can have an area of at least 5 cm squared. The method can also include collecting particles onto the detection surface and measuring the collected particles.

In accordance with another embodiment of the present disclosure, a sensor assembly is described. The sensor assembly can include a source portion having a housing, the source portion being configured to generate an optical beam. The sensor assembly can also include a detect portion having a housing spaced from the housing of the source portion. The housing of the detect portion can define a detection surface for receiving the generated optical beam. The sensor assembly can also include a wiper member coupled to the detect portion. The wiper member can be movably coupled to the detection portion such that the wiper member can move between at least a first position, where the wiper member surrounds the detection surface, and a second position, where the wiper member is spaced apart from the detection surface.

In any of the embodiments described herein, the sensor assembly can include a second housing portion spaced apart from the first housing portion. The second housing portion can be configured for directing a beam towards the first housing portion.

In any of the embodiments described herein, the beam can be directed to the detection surface of the first housing portion.

In any of the embodiments described herein, the beam can be an expanding beam.

In any of the embodiments described herein, the beam can define a cross-sectional area, and the cross-sectional area of the beam can cover at least the entire area of detection surface.

In any of the embodiments described herein, the detection surface can be substantially planar.

In any of the embodiments described herein, the detection surface can be positioned substantially centrally along a surface of the first housing portion.

In any of the embodiments described herein, the optical beam can be an expanding optical beam.

In any of the embodiments described herein, the optical beam can define a cross-sectional area, and the cross-sectional area of the beam can cover at least the entire area of detection surface.

In any of the embodiments described herein, the sensor assembly can include a chassis having a first end coupled to the source portion and a second end coupled to the detect portion.

In any of the embodiments described herein, at least a portion of the wiper member can be configured to move across the detection surface when the wiper member transitions from the first position to the second position.

In any of the embodiments described herein, the wiper member can define a body having a cylindrical shape.

In any of the embodiments described herein, the wiper member can define a body having a hollow shape.

In any of the embodiments described herein, the optical beam can reach the detection surface when the wiper member is in the first position.

In any of the embodiments described herein, the detection surface can have an area of at least 5 cm squared.

### DESCRIPTION OF THE DRAWINGS

The foregoing aspects and many of the attendant advantages of this disclosure will become more readily appreciated by reference to the following detailed description, when taken in conjunction with the accompanying drawings, wherein:

FIG. 1 illustrates a perspective view of an exemplary submersible system in accordance with one or more embodiments of the present disclosure;

FIG. 2 illustrates a perspective view of an exemplary sensor assembly in accordance with one or more embodiments of the present disclosure;

FIG. 3 illustrates a cross-sectional view of the sensor assembly of FIG. 2;

FIG. 4 illustrates a cross-sectional view of a detection portion in accordance with one or more embodiments of the present disclosure;

FIG. 5 illustrates a top view of the sensor assembly of FIG. 2;

FIG. 6 illustrates a partial perspective view of a sensor assembly including a wiper assembly in accordance with one or more embodiments of the present disclosure;

FIGS. 7A and 7B illustrate perspective views of a wiper assembly transitioning from a first position to a second position in accordance with one or more embodiments of the present disclosure; and

FIG. 8 is a flow diagram illustrating an example method for operating a sensor assembly in accordance with one or more embodiments of the present disclosure.

### DETAILED DESCRIPTION

As more and more greenhouse gases are released into the atmosphere, understanding how these gases impact the oceanic carbon cycle, including the biological carbon pump, may help in the fight against climate change. Measuring changes in gravitational carbon particle flux (i.e., change in sinking carbon particles over time) within the ocean can be a helpful tool in understanding the impact greenhouse gases are having with oceanic carbon cycle. However, due in part to the vastness of the ocean, obtaining reliable data on carbon particle flux can be a challenging undertaking. Utilizing autonomous robotic ocean floats may help with obtaining data, as these ocean floats can be deployed across the world. However, conventional sensors for measuring gravitational carbon particle flux are not suited for use with ocean floats or other ocean profilers. Accordingly, there exists a need for a sensor that can reliably measure carbon particles and be used with an ocean float, submersible, or other ocean profiler.

Embodiments of the present disclosure can address these and other issues of using sensors with ocean floats (or other ocean profilers). In some examples, embodiments of the present disclosure relate to a sensor assembly that can be used to measure carbon particles within the ocean. This sensor assembly can be coupled to a suitable submersible (such as an ocean float) so that the sensor assembly can be deployed along with the submersible. Once deployed, the sensor assembly takes measurements to help determine the amount of gravitational carbon particle flux within the water. As will be described in more detail herein, the sensor assembly can be suited for use with a submersible. For instance, as one example, the sensor assembly can utilize a suitably sized detection surface to collect carbon particles reliably and passively as the submersible is deployed. As another example, the sensor assembly can self-clean, which can increase the time the sensor assembly can remain operational without needing to retrieve the sensor assembly. These and other aspects of the present disclosure will be more fully described below.

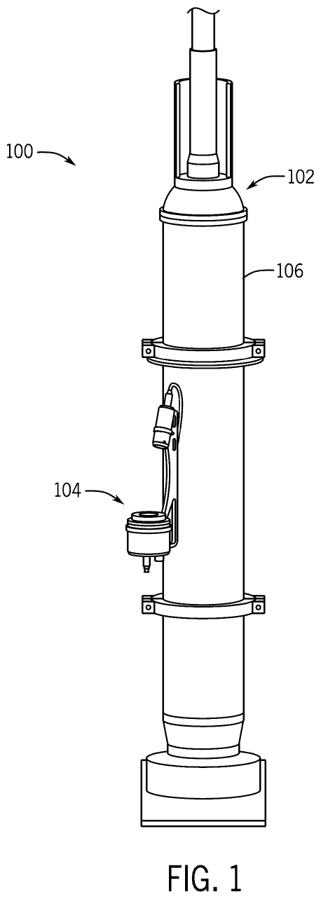

FIG. 1 illustrates a perspective view of a submersible system 100 in accordance with exemplary embodiments of the present disclosure. In general, the submersible system 100 can include a submersible vehicle 102 and a sensor assembly 104 coupled to the submersible vehicle 102. The submersible vehicle 102 can be any suitable vehicle for use in water, including, for instance, an ocean profiling float, an underwater glider, or other underwater vehicle. The sensor assembly 104 can be used for collecting data, including data about gravitational carbon particle flux and/or other properties. For instance, as will be described in more detail herein, the sensor assembly 104 can be configured to measure the concentration of sinking particles (e.g., sinking carbon particles) within the water and how this concentration changes over time. However, in other arrangements, the sensor assembly 104 may be configured to measure a different property, including, for example, the concentration of other particles besides carbon.

The sensor assembly 104 can be both mechanically and electrically coupled to the submersible vehicle 102. In some arrangements, the sensor assembly 104 can be mechanically coupled to an outer housing 106 of the submersible vehicle 102, as shown in FIG. 1. In other arrangements, however, the sensor assembly 104 may be coupled to another suitable location. For instance, the sensor assembly 104 may be coupled to an internal frame of the submersible vehicle 102. The sensor assembly 104 can be electrically coupled with the electronics of the submersible vehicle 102 through any suitable electrical connections, including wiring. Arranging the sensor assembly 104 in this manner can allow for the sensor assembly 104 to interact with the electronics of the submersible vehicle 102 for various operational purposes, including, for instance, to draw power from the submersible vehicle 102 and, in some embodiments, to transmit data via the communications system of the submersible vehicle 102.

While the illustrated embodiment shows the submersible system 100 as including a single sensor assembly 104 it should be appreciated that the submersible system 100 may include additional sensor assemblies 104 coupled to the submersible vehicle 102. Additionally, in some embodiments, the sensor assembly 104 may be used independently from the submersible system 100 and/or submersible vehicle 102. For instance, as one non-limiting example, the sensor assembly 104 may be coupled to a different vessel or ocean-observing platform, or the sensor assembly 104 may be used independently of any external vessel or platform. These and other arrangements of the submersible system 100 and sensor assembly 104 are within the scope of the present disclosure.

As previously stated, the sensor assembly 104 can be configured for collecting data on carbon particles, including sinking carbon particles, within water. In one example embodiment, the sensor assembly 104 can be configured to collect carbon particles passively within the water and measure the amount of collected carbon particles. For instance, the sensor assembly 104 can be positioned so that at least a portion of the sensor assembly 104 can collect carbon particles as the particles naturally sink within the water. The sensor assembly 104 can then measure the collected particles through any suitable methods, including, for instance, a suitable optical measurement. In some arrangements, the sensor assembly 104 can collect data on how the carbon particles change over time. For example, the sensor assembly 104 can periodically measure the amount of collected carbon particles and save each measurement as a separate data point for comparison. These resulting measurements can help provide insight into the amount of sinking carbon particles that are present in a particular portion of water.

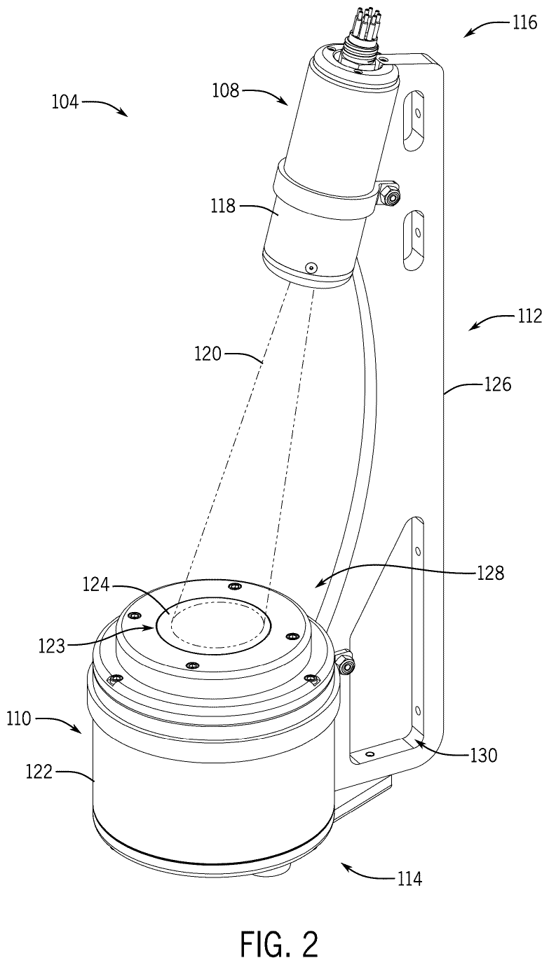

FIG. 2 illustrates a perspective view of an example sensor assembly 104 in accordance with one or more embodiments of the present disclosure. As shown in FIG. 2, the sensor assembly 104 can include a source portion 108 and a detect portion 110 that are coupled together by a chassis 112. The source portion 108 can be configured to generate a beam 120 for detecting carbon particles and can be positioned at or near a first end 114 of the sensor assembly 104. The detect portion 110 can be configured to receive the generated beam 120 and can also be configured to capture carbon particles for detection by the source portion 108. The detect portion 110 can be positioned at or near a second end 116 of the sensor assembly 104. The chassis 112 can be configured to support both the source portion 108 and detect portion 110 and can provide a suitable platform for coupling the sensor assembly 104 to an external object (e.g., the submersible vehicle 102). As will be described in more detail herein, these components of the sensor assembly 104 can work together to measure gravitational carbon particle flux within a body of water.

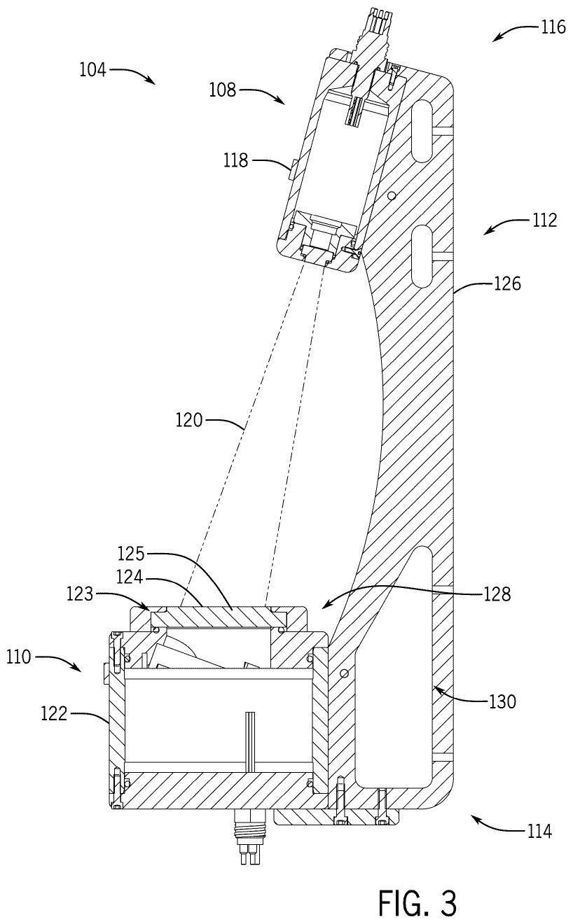

FIG. 3 illustrates a cross-sectional view of the sensor assembly 104 from FIG. 2. Referring to FIG. 3, the source portion 108 can include a housing 118 for housing one or more electrical and optical components, such as an LED source and lens. These components within the housing 118 can be arranged so that the beam 120 can be generated and directed out of the housing 118, as shown in FIGS. 2 and 3. In some arrangements, and as will be described in more detail herein, the beam 120 can be used to detect the presence of carbon particles that are collected by the sensor assembly 104. In some of these arrangements, or otherwise, the beam 120 can be an expanding beam that is directed to the detect portion 110. For example, the beam 120 can have a cross-sectional area that increases in size as the beam 120 travels away from the source portion 108 and towards the detect portion 110.

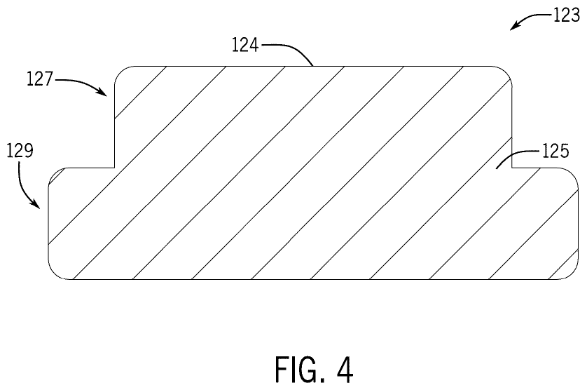

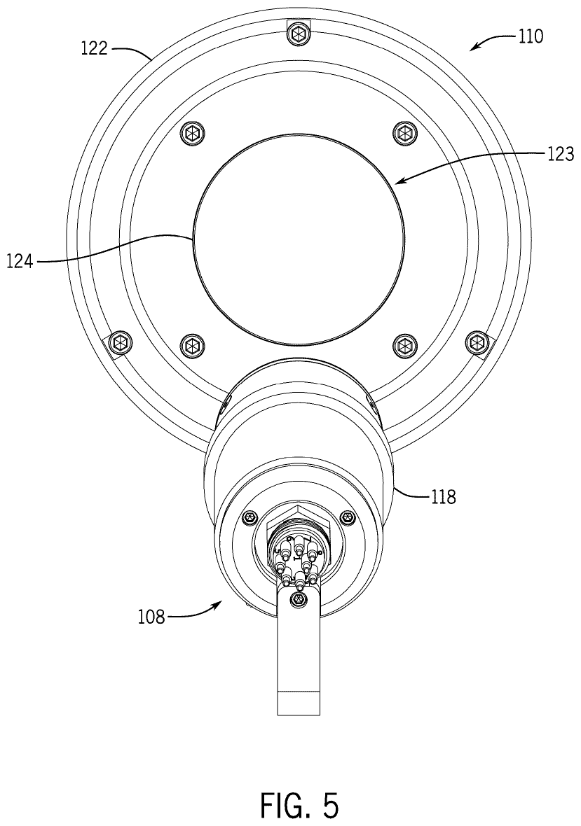

FIG. 4 illustrates a cross-sectional view of a detection portion 123 that can be disposed within the detect portion 110, and FIG. 5 illustrates a top view of the sensor assembly 104 from FIG. 2. Referring to FIGS. 2 to 5 together, the detect portion 110 can include a housing 122 for housing various components of the sensor assembly 104, including the detection portion 123 which can be coupled to the housing 122. The detection portion 123 can define a detection surface 124 that can be externally exposed along at least a portion of the housing 122. As will be described in more detail below, the detection surface 124 can be configured to capture (or collect, trap, etc.) carbon particles for analysis by the sensor assembly 104. Accordingly, this detection surface 124 can be positioned in view of the source portion 108 such that the generated beam 120 can reach and be received by the detection surface 124. In some of these arrangements, the beam 120 can cover the entire area of the detection surface 124. Stated differently, in some examples, the cross-sectional area of the beam 120 is at least the same size as the area of the detection surface 124. In various arrangements, the detection portion 123 can be formed from a window-like material that allows for the beam 120 to travel through the detection portion 123. Arranging the detection portion 123 in this manner can allow for the beam 120 to be received and analyzed by the detect portion 110. In some of these arrangements or otherwise, the detect portion 110 can record images for analysis (e.g., record images of the detection surface 124, record the light intensity on a photodiode, etc.). Accordingly, in some embodiments, the housing 122 of the detect portion 110 can house one or more electrical/optical components, including, for instance, lenses, mirrors, and diffusers, which can be used to receive and analyze a beam, or to record images or data.

As previously noted, the detection portion 123 can be configured to capture sinking particles within the water on the detection surface 124, including sinking carbon particles. For example, as shown in FIGS. 2 to 5, the detection surface 124 can be externally exposed and can form a substantially planar surface that defines an area for receiving particles. In some of these examples, the detection surface 124 can be positioned substantially centrally along an upper surface of the housing 122. The sensor assembly 104 can also be positioned relative to the submersible vehicle 102 such that the detection surface 124 is oriented upwards towards the surface of the water. Accordingly, by arranging the detection surface 124 as a planar surface and by orienting the detection surface 124 towards the surface of the water, sinking particles within the water can sink directly onto the detection surface 124 for analysis. Thus, in some arrangements, the detection portion 123 can passively collect carbon particles on the detection surface 124 while the sensor assembly 104 is deployed.

A brief, example operation of the sensor assembly 104 will now be described with reference to FIGS. 1 to 5. With the sensor assembly 104 coupled to a submersible vehicle 102, the submersible system 100 can be deployed to a desired location within a body of water. In some embodiments, the submersible vehicle 102 can dive to a desired depth and remain at that depth for a length of time. In some of these embodiments, the submersible vehicle 102 can float with the water currents at the desired depth (e.g., the submersible vehicle moves with the currents but remains at the desired depth). With the submersible vehicle 102 deployed to a desired depth, the detect portion 110 of the sensor assembly 104 can begin collecting carbon particles as the carbon particles sink within the body of water. Periodically, the sensor assembly 104 can measure the collected carbon particles and record the resulting data. In some arrangements, the sensor assembly 104 measures the collected carbon particles by generating a beam 120, directing the beam towards the detection surface 124, and measuring the resulting beam and/or diffuse attenuation. The recorded data can be transmitted from a communication system on submersible vehicle 102 or can be saved and later retrieved when the submersible system 100 returns from deployment.

Additional features of the sensor assembly 104 will now be described. Referring again to FIG. 4, the detection portion 123 can have a body 125 defining a first, upper portion 127 and a second, lower portion 129, with the detection surface 124 being defined along the upper surface of the upper portion 127. In some arrangements, and as shown in FIG. 4, the upper portion 127 can be sized differently from the lower portion 129. For example, in the illustrated embodiment, the upper portion 127 is smaller than the lower portion 129. Arranging the detection portion 123 in this manner can allow for the detection portion 123 to interface with the housing 122 without impacting the optics of the sensor assembly 104. For instance, the detection portion 123 can be clamped along outer edge of the lower portion 129 which, due to the increased size of the lower portion 129, can limit any impact the clamping may have on the beam 120 (e.g., through shading). Additionally, the stepped nature between the upper and lower portion 127, 129 can allow for a seal (e.g., an O-ring) to be placed between the lower portion 129 and the housing 122, which can seal the inside of the housing 122 from water when the sensor assembly 104 is in use.

In some embodiments, the size and shape of the detection portion 123 may impact performance of the sensor assembly 104. As one example of this impact, the size and shape of the detection surface 124 can affect the accuracy of the sensor assembly 104. Carbon (and other) particles within the water can come in a variety of different sizes. If these carbon particles are of similar size to the detection surface 124 (or are larger than the detection surface 124) the sensor assembly 104 may be unable to distinguish between the presence of one or many carbon particles. Additionally, if these carbon particles get dislodged from the detection surface 124, those dislodged particles can greatly (and potentially negatively) affect the accuracy of the resulting measurements. Accordingly, ensuring the detection surface 124 has a suitably sized area can limit issues that may arise due to the size of carbon particles or carbon particles being dislodged. Thus, in some arrangements, the detection surface 124 can have an area that is between 1 cm squared to about 50 cm squared. In some examples, the detection surface 124 can have an area that is at least 5 cm squared, at least 25 cm squared, at least 30 cm squared, or some other size. In some of these examples, or otherwise, the detection surface 124 can define a circular area having a diameter of about 5 cm or greater.

In some specific instances, it may be desirable to include a detection surface 124 having an area that is at least 5.7 cm squared. In some of these specific instances, or otherwise, decreasing the area below 5.7 cm squared may decrease the accuracy of any measurements, as the size of collected particles may be too similarly sized to the resulting area of the detection surface 124. In other instances, it may be desirable to include a detection surface having an area of about 30.8 cm squared. Arranging the detection surface 124 in this, or in any of these manners, can ensure that many carbon particles can be captured and individually measured by the sensor assembly 104 without negatively affecting the resulting data.

To limit interference from coupling the detection portion 123 to the housing 122, in some arrangements, the lower portion 129 can have a diameter that is at least 0.5 cm larger than the diameter of the upper portion 127. In some examples, the diameter of the lower portion 129 can be at least 2 cm larger than the diameter of the upper portion 127. In various examples, the diameter of the lower portion 129 can be about 8.3 cm.

With the detection surface 124 having an increased area (e.g., an area greater than 1 cm squared), in some instances, the beam 120 generated by the source portion 108 may also need to have an increased cross-sectional area. In some of these instances, or otherwise, the beam 120 can have a cross-sectional area that is substantially the same area as the area of the detection surface 124. Additionally, in some embodiments, the beam 120 can be an expanding beam such that the cross-sectional area of the beam 120 is at least the same area of the detection surface 124 when the beam 120 reaches the detection surface 124. Matching the cross-sectional area of the beam 120 with the area of the detection surface 124 can help ensure the sensor assembly 104 accurately measures the collected carbon particles on the detection surface 124.

In some embodiments, selecting a suitable thickness for the detection portion 123 can improve the performance of the sensor assembly 104. For example, in arrangements where the beam 120 travels through detection portion 123, minimizing the thickness of the detection portion 123 can improve the signal to noise ratio. In some arrangements, the thickness of the detection portion 123 can be between about 1 cm to about 5 cm. In some examples, the thickness of the detection portion 123 is at least 1.27 cm, with the thickness being evenly divided between the upper and lower portions 127, 129.

While reducing the thickness of the detection portion 123 can improve performance, the detection portion 123 may still require enough robustness to withstand conditions within the ocean (e.g., pressure, salinity, etc.). In some arrangements, selecting a suitable material for the detection portion 123 can improve the strength of the detection portion 123 without necessarily needing to increase the thickness or weight. For instance, forming the detection portion 123 as a sapphire window can allow for the detection portion 123 to have sufficient strength to withstand ocean pressures at depths of at least 2000 meters even at a thickness of about 1 cm.

In some embodiments, the positioning of sensor assembly 104 components may shade a portion of the detection surface 124, which can prevent the sensor assembly 104 from getting reliable results. For example, the housing 118 for the source portion 108 can be positioned above the detection surface 124 such that housing 118 may disrupt sinking particles and direct those particles away from detection surface 124 (e.g., through colliding with the housing 118, turbulence, etc.). This disruption may lead to inaccurate readings, as the sensor assembly 104 may be unable to reliably account for the disrupted particles. To limit issues resulting from shading, the housing 118 of the source portion 108 can be positioned offset from the detection surface 124. For instance, as shown in FIG. 5, the housing 118 of the source portion 108 can be positioned away from the detection surface 124 such that a lateral gap exists between the housing 118 of the source portion 108 and the detection surface 124. Positioning the housing 118 of the source portion 108 in this manner can result in an unobstructed detection surface 124 with limited disruption from the housing 118. Stated differently, in some arrangements, carbon particles can sink onto the detection surface 124 without interference from the housing 118 of the source portion 108. To account for the offset arrangement of the source portion 108, the generated beam 120 may need to be projected towards the detection surface 124 at a non-zero angle. In some of these arrangements, or otherwise, the sensor assembly 104 may need to be calibrated to account for the angled beam 120.

In some instances, maintaining the positioning and orientation of the source portion 108 relative to the detect portion 110 can improve the reliability and accuracy of the sensor assembly 104. For example, in embodiments where the beam 120 is an expanding beam, maintaining the positioning of the source portion 108 relative to the detect portion 110 can help ensure the beam 120 reaches the detection surface 124 with the appropriate cross-sectional area. Accordingly, in some arrangements, the chassis 112 can help maintain the positioning and orientation of the source portion 108 and detect portion 110. Referring to FIG. 3, the chassis 112 can include a body 126 that extends between the first and second ends 114, 116 of the sensor assembly 104 such that the source portion 108 couples to the body 126 at or near the first end 114 and the detect portion 110 couples to the body at or near the second end 116. The body 126 can have a suitable thickness and can be formed from a suitable material such that the chassis 112 can withstand the operating conditions of the submersible vehicle 102 (e.g., ocean pressure, salinity, etc.) while maintaining the positioning and orientation of the source portion 108 relative to the detect portion 110.

The chassis 112 can include additional features that improve the functionality and ease of use of the sensor assembly 104. As one example, and as shown in FIG. 3, the chassis 112 can define a gap 128 that extends around the circumference of the detection surface 124. Forming this gap 128 can reduce turbulence from the chassis 112 around the detection surface 124. As another example, the chassis 112 can include one or more coupling portions 130 that are formed along the length of the chassis 112. These coupling portions 130 can allow for the sensor assembly 104 to be easily coupled to a separate object (e.g., the submersible vehicle 102) through a suitable fastener (e.g., a hose clamp).

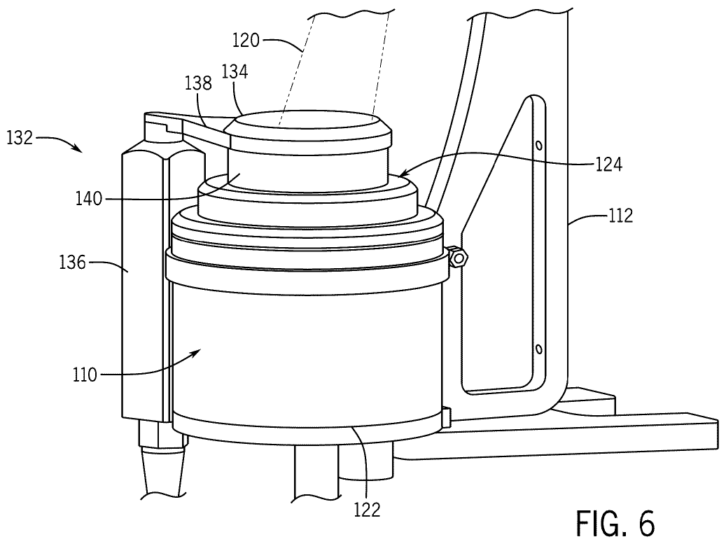

In some arrangements, it may be desirable to provide protection to the area surrounding the detection surface 124. For instance, in some situations, debris may block or obstruct a portion of the beam 120, which can lead to inaccurate results. In other situations, debris may remove particles off the detection surface 124, which can also lead to inaccurate results. Separately, in some scenarios, it may be desirable to clean the detection surface 124 without needing to manually retrieve the submersible system 100. For example, while the submersible system 100 remains deployed, it may be desirable to rerun testing for carbon particles with a cleaned detection surface 124. To address these and other issues with the sensor assembly 104, in some embodiments, a wiper assembly 132 can be employed (see FIG. 6). As will be described in more detail herein, the wiper assembly 132 can be configured to limit debris (or other objects, ocean currents, etc.) from interfering with operation of the sensor assembly 104. Furthermore, the wiper assembly 132 can be configured to clean the detection surface 124 when desired and without needing to manually retrieve the submersible system 100. These and other aspects of the present disclosure will be more fully described below.

FIG. 6 illustrates a perspective view of a detect portion 110 including a wiper assembly 132 in accordance with one or more embodiments of the present disclosure. As shown in FIG. 6, the wiper assembly 132 can be coupled to the housing 122 of the detect portion 110. The wiper assembly 132 can include a wiper member 134 positioned over the detection surface 124, a motor 136 coupled to the housing 122, and an arm 138 extending between the wiper member 134 and motor 136 to couple the wiper member 134 to the motor 136. Together, these components can be configured to both limit debris from interfering with operation of the sensor assembly 104 and to clean the detection surface 124 when desired.

The wiper member 134 can be configured to protect the detection surface 124 from debris, or limit debris from reaching the detection surface 124. For example, as shown in FIG. 6, the wiper member 134 can define a cylindrical hollow body 140 that can surround the detection surface 124. This body 140 can contact the housing 122 of the detect portion 110 and extend off the housing 122 a desired distance. By arranging the wiper member 134 in this manner, the wiper member 134 surrounds the detection surface 124, which can limit undesired debris from flowing into the space surrounded by the wiper member 134. This arrangement can still allow for carbon particles to sink onto the detection surface 124, as the carbon particles can sink through the hollow portion of the body 140. Accordingly, this arrangement can limit undesired debris from interfering with the operation of the sensor assembly 104 (e.g., due to debris blocking the beam 120, debris blocking particles, debris removing particles, etc.). In some examples, and as shown in the illustrated embodiment, the height of the body 140 can be such that the wiper member 134 provides protection to the detection surface 124 but does not block the beam 120 itself (e.g., the beam 120 can travel through the hollow portion of the body 140 unimpeded by the body 140). Thus, in some of these examples, or otherwise, the wiper member 134 can be positioned over the detection surface 124 without directly interfering with the operation of the sensor assembly 104.

The wiper member 134 can also be configured to clean the detection surface 124. To clean the detection surface, the motor 136 can move the wiper member 134 across the detection surface 124 to thereby clear the detection surface 124 of any collected particles or debris. Accordingly, in some arrangements, the wiper member 134 can be positioned such that at least a portion of the wiper member 134 contacts the detection surface 124 when the wiper member 134 is moved across the detect portion 110. Additionally, in some of these examples, or otherwise, the wiper member 134 can be formed from a material that is suited for cleaning a surface (e.g., rubber, plastic, etc.). In some instances, the wiper member 134 can be formed as a brush, including as a cylindrical brush with a hollow center.

While the illustrated embodiment shows wiper assembly 132 as including a wiper member 134, motor 136, and arm 138, it should be appreciated that the wiper assembly 132 may include other components or may be arranged in a different manner. As one non-limiting example, in some embodiments, the wiper member 134 can include perforated walls such that the wiper member 134 does not limit the flow of water across the detection surface 124. As another non-limiting example, the wiper member 134 may be arranged exclusively for cleaning the detection surface 124 and removing debris. For instance, the wiper member 134 may have an increased length such that the wiper member 134 could remove debris that is located closer to the source portion 108 but the wiper member 134 would otherwise interfere with the beam 120 if positioned over the detection surface 124. As another example, the wiper member 134 can remain fixed over the detection surface 124. As another example, the wiper assembly 132 may exclude the arm 138 such that the motor 136 directly couples to the wiper member 134, These and other arrangements of the wiper assembly 132 are within the scope of the present disclosure.

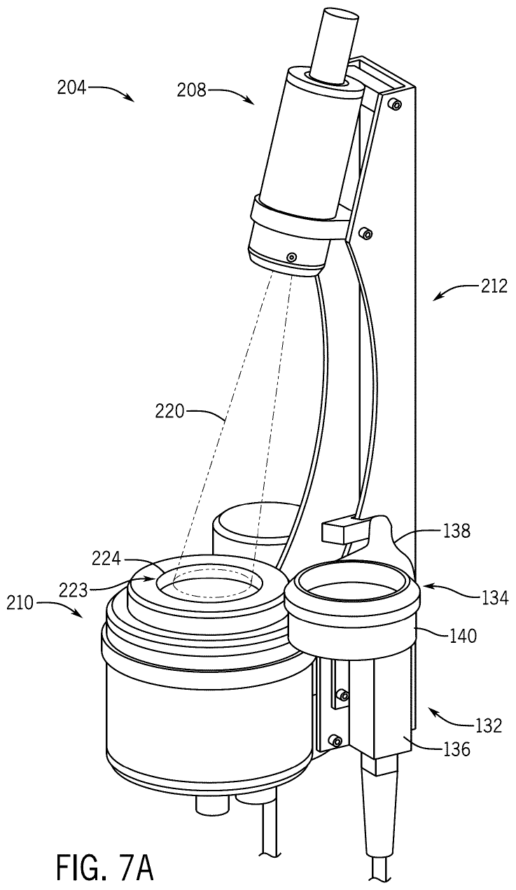

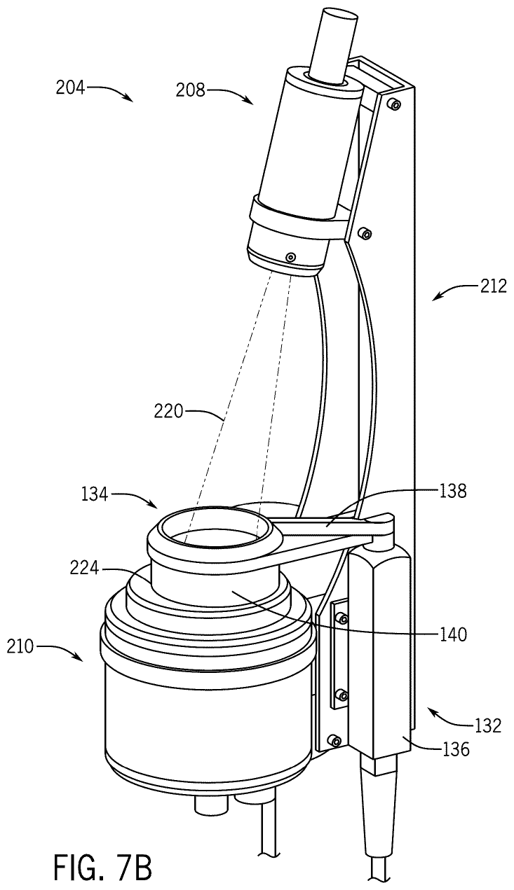

FIGS. 7A and 7B illustrate a sensor assembly 204 including a wiper assembly 132. The sensor assembly 204 can be generally similar to the sensor assembly 104, where like numerals indicate like components. For example, the sensor assembly 204 can include a source portion 208 and a detect portion 210, which can be generally similar (or identical) to the source portion 108 and detect portion 110 of the sensor assembly 104. Accordingly, the sensor assembly 204 can include similar (or identical) components as the sensor assembly 104 and can function similar (or identical) to the sensor assembly 104, unless indicated otherwise herein.

As shown in FIGS. 7A and 7B, the wiper assembly 132 can be movable relative to the detect portion 210 between two or more positions. For instance, the wiper assembly 132 can be movable between at least a first position, where the wiper member 134 is clear of the detection portion 223 and detection surface 224, and a second position where the wiper member 134 is positioned above the detection portion 223 and surrounds the detection surface 224. FIG. 7A shows the wiper assembly 132 in the first position. When in the first position, the wiper assembly 132 is coupled to the chassis 212 with the wiper member 134 extending outwards therefrom. The wiper member 134 can be spaced away from the detection surface 224, which can ensure that the wiper member 134 does not interfere with the detection surface 224 or beam 220 during operation. FIG. 7B shows the wiper assembly in the second position. When in the second position, the wiper assembly 132 remains coupled to the chassis 212, but the wiper member 134 is positioned above the detection surface 224 such that the body 140 of the wiper member 134 surrounds the outer perimeter of the detection surface 224. In this position, the wiper member 134 can provide protection to the detection surface 224 while also allowing for the beam 220 to reach the detection surface 224 without any interference from the wiper assembly 132.

To transition the wiper assembly 132 from the first position to the second position, the motor 136 can rotate the wiper member 134 through its connection with the arm 138 until the wiper member 134 is at the desired position. In some examples, this movement of the wiper member 134 (e.g., transitioning the wiper member 134 from the first position to the second position) can act to clean the detection surface 224 or clear debris obstructing the beam 220 from reaching the detection surface 224. For example, the base of the wiper member 134 can scrape the detection surface 224 as the wiper member 134 is moved across the detection surface 224 and/or can push debris out of the path of the beam 220.

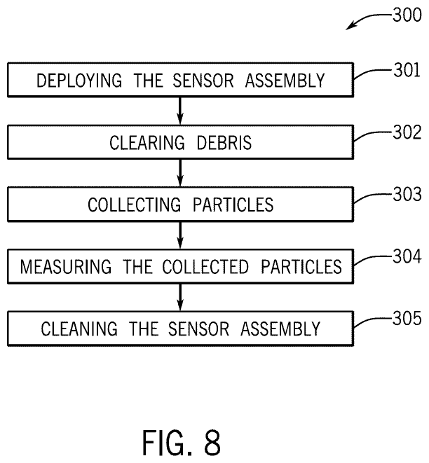

FIG. 8 is a flow diagram illustrating an example method 300 of operating a sensor assembly. The method 300 can be used with any of the sensor assemblies described herein, including the sensor assembly 104 and the sensor assembly 204.

At step 301, the method 300 can begin with deploying the sensor assembly. To deploy the sensor assembly, the sensor assembly can first be coupled to a suitable vehicle (e.g., the submersible vehicle 102). Once coupled to a suitable vehicle, the vehicle can be deployed to a desired body of water, and, in some cases, dive to a desired depth for taking measurements.

At step 302, the method 300 can optionally include clearing debris. With the vehicle and sensor assembly in position, the sensor assembly can clear debris that may be blocking its source portion (e.g., the source portion 108) or detect portion (e.g., the detect portion 110). In some examples, the debris can be cleared using a wiper assembly (e.g., the wiper assembly 132). For instance, the wiper assembly can swing a wiper member (e.g., the wiper member 134) across the detect portion to remove any potential obstructions.

At step 303, the method 300 continues with collecting particles. With the vehicle and sensor assembly in position, and the sensor assembly optionally clear of any debris, the sensor assembly can begin collecting particles for measurements. In some arrangements, collecting particles includes having the sensor assembly remain in position within the water over a period of time. As the sensor assembly remains in position, particles (such as carbon particles) may sink onto the sensor assembly, including onto the detection surface (e.g., the detection surface 124) of the detect portion.

At step 304, the method 300 continues with measuring the collected particles. After a desired amount of time has passed, the sensor assembly may measure the number of particles that have been collected on the detection surface. In some arrangements, the sensor assembly can measure the number of particles by directing a beam (e.g., the beam 120) onto the detection surface and measuring the beam attenuation and/or diffuse attenuation. This beam can be an expanding beam with a cross-sectional area that substantially matches the area of the detection surface. Once the number of particles have been measured, this data can be stored or transmitted as desired.

At step 305, the method 300 can optionally include cleaning the sensor assembly. After the sensor assembly has completed its measurements (or, in some instances, before the sensor assembly has begun making measurements), the sensor assembly may clean the detection surface. In some arrangements, the detection surface can be cleaned by the wiper assembly. For example, the wiper assembly can swing the wiper member across the detection surface to remove any particles on the detection surface.

While the concepts of the present disclosure are susceptible to various modifications and alternative forms, specific embodiments thereof have been shown by way of example in the drawings and are described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Language such as “top”, “bottom”, “vertical”, “horizontal”, “lateral”, etc. in the present disclosure is meant to provide orientation for the reader with reference to the drawings and is not intended to be the required orientation of the components or to impart orientation limitations into the claims. Where appropriate from the context, language used to denote an approximation, such as “approximately”, “substantially”, “about”, “near”, etc. can refer to standard engineering tolerances. Additionally, or alternatively, where appropriate, language used to denote an approximation can refer to plus or minus 1%, 5%, 10%, or 15% of the described or implied value.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, it may not be included or may be combined with other features.

1. A sensor assembly, comprising:
a source portion having a housing, wherein the source portion is configured to generate an optical beam;
a detect portion having a housing spaced from the housing of the source portion, wherein the housing of the detect portion defines a detection surface for receiving the generated optical beam; and
a wiper member coupled to the detect portion, wherein the wiper member is movably coupled to the detect portion such that the wiper member can move between at least a first position, where the wiper member surrounds the detection surface, and a second position, where the wiper member is spaced apart from the detection surface.

2. The sensor assembly of claim 1, wherein at least a portion of the wiper member is configured to move across the detection surface when the wiper member transitions from the first position to the second position.

3. The sensor assembly of claim 1, wherein the wiper member defines a body having a cylindrical shape.

4. The sensor assembly of claim 1, wherein the wiper member defines a body having a hollow shape.

5. The sensor assembly of claim 4, wherein the optical beam can reach the detection surface when the wiper member is in the first position.

6. The sensor assembly of claim 1, wherein the detection surface has an area of at least 5 cm squared.

7. A submersible system, comprising:
a submersible vehicle having a housing;
a sensor assembly coupled to the housing of the submersible vehicle, the sensor assembly including a housing portion, wherein the housing portion defines a detection surface for collecting particles; and
a wiper member coupled to the sensor assembly, wherein the wiper member is configured to move between at least a first position, where the wiper member surrounds the detection surface, and a second position, where the wiper member is spaced apart from the detection surface.

8. The submersible system of claim 7, wherein at least a portion of the wiper member is configured to move across the detection surface when the wiper member transitions from the first position to the second position.

9. The submersible system of claim 7, wherein the wiper member defines a body having a cylindrical shape.

10. The submersible system of claim 7, wherein the wiper member defines a body having a hollow shape.

11. The submersible system of claim 10, wherein the sensor assembly is configured to generate an optical beam, and wherein the generated optical beam can reach the detection surface when the wiper member is in the first position.

12. The submersible system of claim 11, wherein the detection surface has an area of at least 5 cm squared.

13. A method of operating a sensor assembly, the method comprising:
coupling a sensor assembly to a submersible vehicle, wherein the sensor assembly includes:
a source portion having a housing, wherein the source portion is configured to generate an optical beam;
a detect portion having a housing spaced from the housing of the source portion, wherein the housing of the detect portion defines a detection surface for receiving the generated optical beam; and
a wiper member coupled to the detect portion, wherein the wiper member is movably coupled to the detect portion such that the wiper member can move between at least a first position, where the wiper member surrounds the detection surface, and a second position, where the wiper member is spaced apart from the detection surface;
deploying the submersible vehicle;
collecting, with the detection surface, particles;
measuring, with the sensor assembly, the particles collected by the detection surface; and
moving the wiper member from the first position to the second position.

14. The method of claim 13, wherein moving the wiper member from the first position to the second position includes moving the wiper member across at least a portion of the detection surface.

15. The method of claim 13, wherein the detection surface collects the particles passively.

16. The method of claim 13, wherein the wiper member is moved to the second position before collecting particles with the detection surface.

17. The method of claim 13, wherein the wiper member defines a body having a cylindrical shape.

18. The method of claim 13, wherein the wiper member defines a body having a hollow shape.
